(19) United States
(12) Patent Application Publication
(10) Pub. No.: US 2016/0338696 A1
(43) Pub. Date: Nov. 24, 2016

(54) SURGICAL NEEDLE HOLDER

(71) Applicant: SOPRANE, Villeurbanne (FR)

(72) Inventor: Thierry LOUBENS, Lyon (FR)

(21) Appl. No.: 15/157,881

(22) Filed: May 18, 2016

Related U.S. Application Data
(60) Provisional application No. 62/162,984, filed on May 18, 2015.

(51) Int. Cl. A61B 17/062; A61B 17/06

(52) U.S. Cl. CPC A61B 17/0625; A61B 17/06066; A61B 17/06109

(57) ABSTRACT

The surgical needle holder (1) includes a cylindrical cannula (2) holding a surgical needle (3) along a longitudinal axis yy′ and in a basically elongated position, which cannula (2) includes a first end presenting a slender profile (2a) which ensures the traversing of the tissues and its penetration to the level of the operating site and includes a second end opposite the first one integral with a gripping handle (2b), a needle pusher (4) introduced inside the cylindrical cannula (2) and retention elements (5) which allow the needle pusher (4) to be temporarily held in a determined position relative to this cylindrical cannula (2).

### FIELD OF THE INVENTION

The present invention relates to a surgical needle holder that allows the tissues to be traversed in order to guide and free a hyper-elastic surgical needle integral with a suture thread in an operating site such as, for example the abdominal cavity of an individual.

### BACKGROUND OF THE INVENTION

The European patent EP 1 274 350 belonging to the applicant teaches an applicator for a surgical needle comprising a sleeve in which the surgical needle is held in a basically elongated position.

The applicator comprises a shaft which penetrates the sleeve in order to allow the extraction of this needle while this sleeve presents at one of its free ends angular indexation means which consists of a conformation following a particular profile which ensures during the extraction of the surgical needle that it is directed in a determined and constant position relative to the main axes of this sleeve.

The applicator for a surgical needle described in the European patent EP 1 274 350 is also intended to cooperate with a previously implanted trocar in order to allow the introduction of this applicator to the level of the operating site.

### SUMMARY OF THE INVENTION

The surgical needle holder according to the present invention has as subject matter to allow surgeons to inject suture needles at the level of the operating site in coelioscopy while avoiding the use of already installed trocars for carrying out the operation.

The surgical needle holder according to the present invention comprises a cylindrical cannula holding a surgical needle along a longitudinal axis yy′ and in a basically elongated position, which cannula comprises a first end presenting a slender profile which ensures the traversing of the tissues and its penetration to the level of the operating site and comprises a second end opposite the first one integral with a gripping handle, a needle pusher introduced inside the cylindrical cannula and retention means that allows the needle pusher to be temporarily held in a determined position relative to this cylindrical cannula.

The surgical needle holder according to the present invention comprises a cylindrical cannula whose slender profile is constituted on part of the circumference of this cannula by at least one inclined section whereas the other part of the circumference of the cannula is formed by a curved profile which approaches the longitudinal axis yy′.

The surgical needle holder according to the present invention comprises a cylindrical cannula whose inclined section of the slender profile has an inclination at an angle β relative to the longitudinal axis yy′ of this cylindrical cannula which is comprised between 10 and 16 degrees.

The surgical needle holder according to the present invention comprises a cylindrical cannula whose curved profile of the slender profile has a radius of curvature R comprised between 7 and 10 millimeters.

The surgical needle holder according to the present invention comprises a cylindrical cannula whose inclined section of the slender profile is realized over its length by at least two inclined sections with different inclinations relative to the longitudinal axis yy′ of this cylindrical cannula.

The surgical needle holder according to the present invention comprises a cylindrical cannula whose first inclined section of the slender profile extends from the peripheral edge of this cylindrical cannula over a length of approximately 3 millimeters and in the direction of the free end of the cannula.

The surgical needle holder according to the present invention comprises a cylindrical cannula whose first inclined section of the slender profile has an inclination of an angle δ relative to the longitudinal axis yy′ of the cylindrical cannula which is comprised between 8 and 10 degrees.

The surgical needle holder according to the present invention comprises a cylindrical cannula whose second inclined section of the slender profile is situated in the extension of the first inclined section and terminating at the level of the free end of this cylindrical cannula.

The surgical needle holder according to the present invention comprises a cylindrical cannula whose second inclined section of the slender profile has an angle Ω relative to the longitudinal axis yy′ of the cylindrical cannula which is comprised between 12 and 16 degrees.

The surgical needle holder according to the present invention comprises a cylindrical cannula whose second inclined section of the slender profile comprises, relative to the longitudinal axis yy′, beveled edges directed in the direction of the outside of this cylindrical cannula in order to constitute a central cutting edge.

The surgical needle holder according to the present invention comprises a cylindrical cannula whose connection between the first and the second inclined section is realized by cutting lateral edges extending in the direction of the center of this cylindrical cannula and more particularly of the cutting central edge.

The surgical needle holder according to the present invention comprises a cylindrical cannula of which each cutting lateral edge of the slender profile is inclined by an angle a comprised between 15 and 20 degrees relative to the longitudinal axis yy′.

The surgical needle holder according to the present invention comprises a cylindrical cannula whose gripping handle comprises two branches extending in two opposite directions and perpendicular to the longitudinal axis yy′.

The surgical needle holder according to the present invention comprises a needle pusher which is constituted by a cylindrical shaft comprising an internal bore which allows, during the placing of the surgical needle in the cylindrical cannula, the housing of the suture thread and of a tappet wheel fixed to one of the ends of this cylindrical shaft.

The surgical needle holder according to the present invention comprises a cylindrical shaft whose internal bore comprises opposite the tappet wheel a free end formed by an internal beveled edge with a conical profile which ensures on the one hand the introduction of the suture thread inside the shaft and on the other hand the guiding of the surgical needle in the axis yy′ of the cylindrical cannula.

### BRIEF DESCRIPTION OF THE DRAWINGS

The following description will allow the invention to be better understood with regard to the attached drawings given by way of non-limiting examples and cites the characteristics which it presents and the advantages which it can obtain:

FIG. 1 is a perspective view showing the surgical needle according to the present invention.

FIGS. 2A-2C are sectional views showing the surgical needle during an extraction of the surgical needle holder according to the present invention.

FIGS. 3 and 4 are detailed views showing the slender profile of the surgical needle holder according to the present invention.

FIGS. 5 and 6 are detailed views showing a variant of the slender profile of the surgical needle holder according to the present invention.

### DETAILED DESCRIPTION OF THE INVENTION

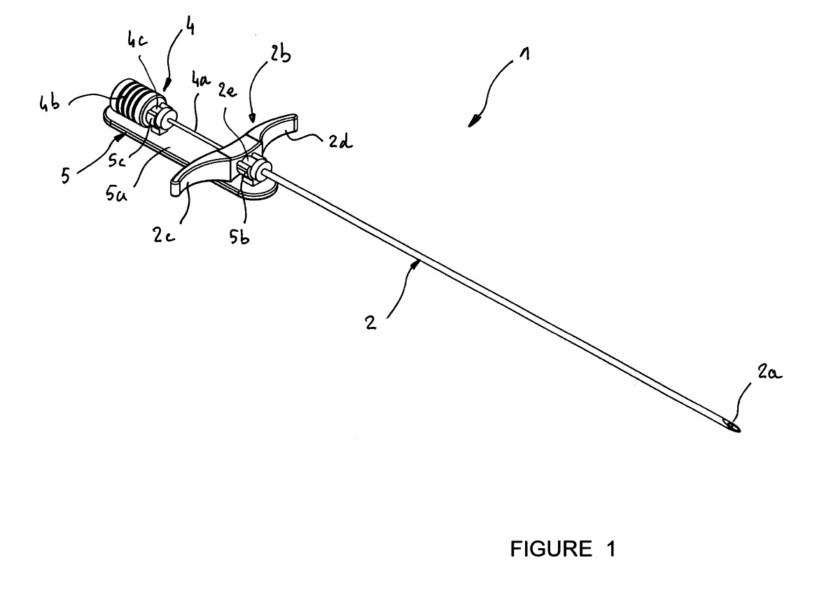

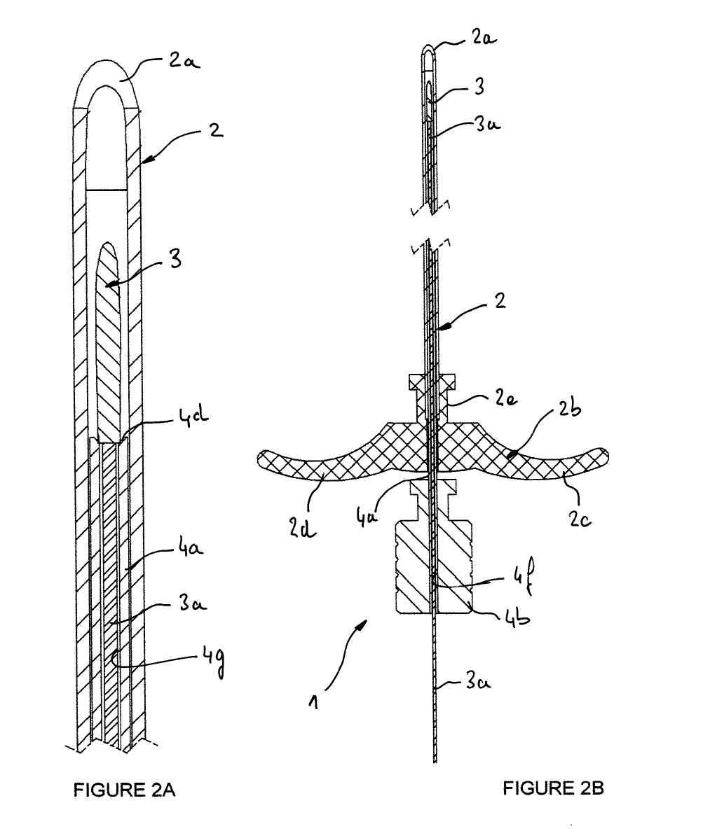

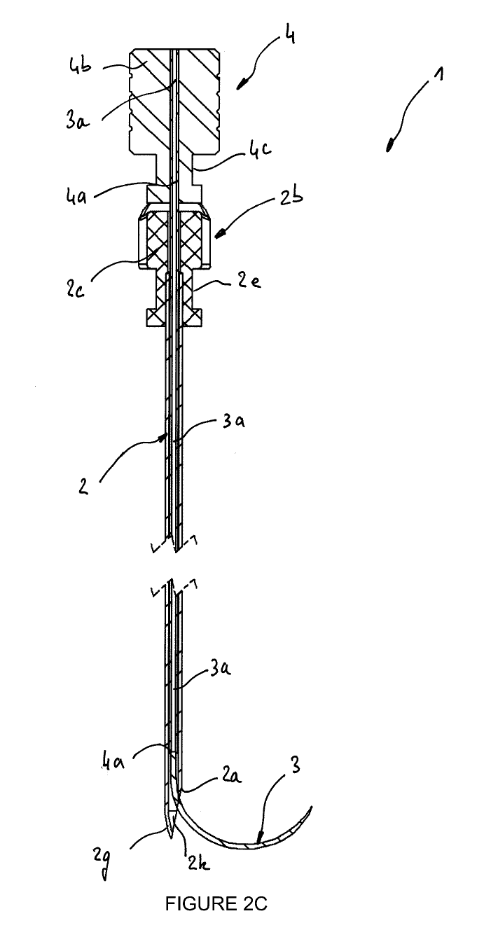

FIGS. 1 and 2A-C show a rechargeable and sterile surgical needle holder 1 comprising a hollow cylindrical cannula 2 inside of which a surgical needle 3 integral with its suture thread 3a is held along a longitudinal axis yy′ in a basically elongated position.

The surgical needle holder 1 comprises a needle pusher 4 introduced inside the cylindrical cannula 2 and retention means 5 that allows the needle pusher 4 to be temporarily maintained in a determined position relative to this cylindrical cannula.

The cylindrical cannula 2 comprises a first end with an opening 2k delimited by a slender profile 2a that ensures the traversing of the tissues of the patient and its penetration at the level of the operating side.

Opposite the slender profile 2a the cylindrical cannula 2 comprises a second end integral with a grasping handle 2b comprising two branches 2c, 2d extending in two opposite directions and perpendicular to the longitudinal axis yy′.

The needle pusher 4 is constituted by a cylindrical shaft 4a integral with one of its ends of a tappet wheel 4b. The tappet wheel 4b comprises an internal bore 4f which allows the passage and the fixing of the cylindrical shaft 4a.

The cylindrical shaft 4a is provided for entering into the inside of the cylindrical cannula 2 in order to allow the extraction of the surgical needle 3 when the surgeon activates the needle pusher 4.

The cylindrical shaft 4a comprises an internal bore 4g that allows, during the placing of the surgical needle 3 in the cylindrical cannula 2, housing the suture thread 3a over the entire length of the needle pusher 4 in order to avoid the effects of jamming and damaging this thread inside this cylindrical cannula 2 (the FIGS. 2A and 2B).

The internal bore 4g of the cylindrical shaft 4a comprises opposite the tappet wheel 4b a free end formed by an internal beveled edge 4d with an optical profile ensuring on the one hand the introduction of the suture thread 3a inside this shaft and on the other hand the guiding of the surgical needle 3 in the axis yy′ of the cylindrical cannula 2 (FIGS. 2A and 2B).

The tappet wheel 4b has a certain weight which permits, when a needle holder 1 is released by the surgeon, that this needle holder comes to rest against the internal wall of the operating site, avoiding any risk of injury that could be caused by the slender profile 2a of the cylindrical cannula 2.

The retention means 5 allows the maintaining of the needle pusher 4 relative to the cylindrical cannula 2 in such a manner that the free end of the shaft 4a opposite the tappet wheel 4b is always supported against the surgical needle 3.

The retention means 5 is constituted by a plate 5a integral with one of its faces of two pincers 5b and 5c forming two fixation clips. Each pincer 5b and 5c cooperates with an impression 4c arranged in the tappet wheel 4b of the needle pusher 4 and with an impression 2e realized in the gripping handle 2b of the cylindrical cannula 2.

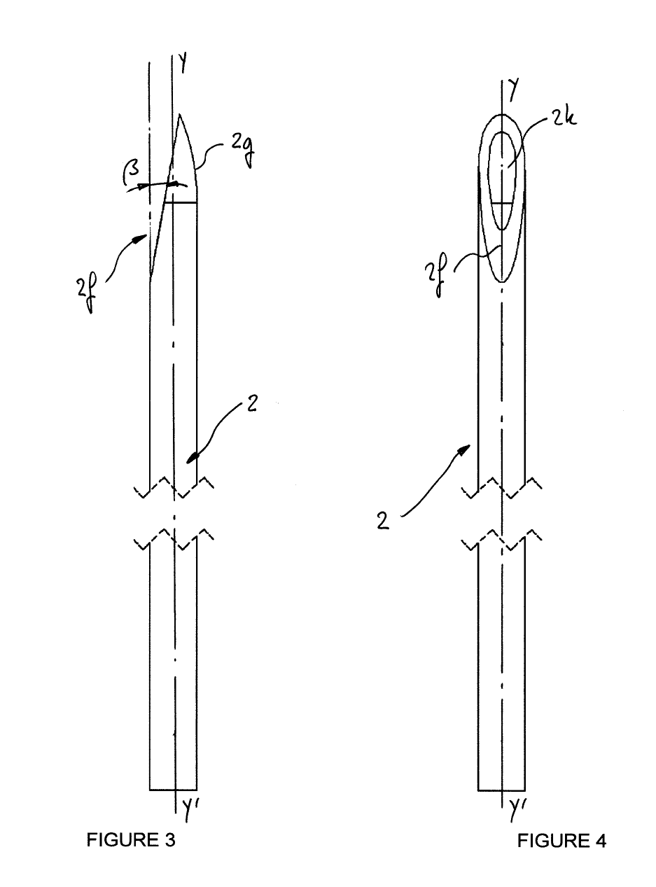

FIGS. 3 and 4 show a first embodiment of the slender profile 2a provided at the level of the first end of the cylindrical cannula 2.

The slender profile 2a is provided to ensure the piercing of the tissues of the patient and the penetration of the cylindrical cannula 2 up to the level of the operating site.

The slender profile 2a of the cylindrical cannula 2 is constituted on a part of the circumference of this cannula and in a horizontal plane parallel to the one containing the branches 2c, 2d of the gripping handle 2b of at least one inclined section 2f while the other part of the circumference of the cannula is formed by a curved profile 2g which approaches the longitudinal axis yy′.

The inclined section 2f has an inclination of an angle β relative to the longitudinal axis yy′ of the cylindrical cannula 2 which is comprised between 10 and 16 degrees.

The curved profile 2g has a radius of curvature R comprised between 7 and 10 millimeters. The curved profile 2g allows reducing the effect of coring during the penetration through the cutaneous tissues of the patient.

It is noted that the inclination of the inclined section 2f and the dimensions of the radius of curvature R of the curved profile 2g position the point of the slender profile 2a of the cylindrical cannula 2 below the longitudinal axis yy′.

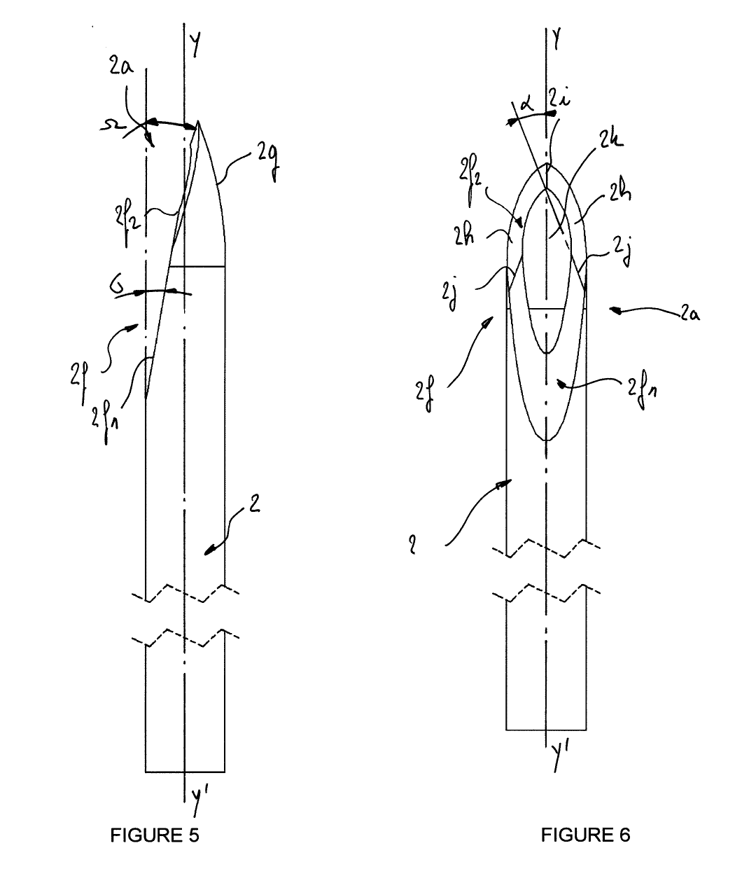

FIGS. 5 and 6 show a second embodiment of the slender profile 2a provided at the level of the first end of the cylindrical cannula 2.

It is noted in this case that the inclined section 2f can be realized along its length by at least two inclined sections 2f₁ and 2f₂ with different inclinations relative to the longitudinal axis yy′ of the cylindrical cannula 2.

Therefore, the first inclined plane 2f₁ extends starting from the peripheral edge of the cylindrical cannula 2 for a length of approximately 3 millimeters and in the direction of the free end of this cannula. This first inclined plane 2f₁ has an inclination of an angle δ relative to the longitudinal axis yy′ of the cylindrical cannula 2 which is comprised between 8 and 10 degrees.

The second inclined section 2f₂ is situated in the extension of the first inclined section 2f₁ and terminates at the level of the free end of the cylindrical cannula 2. The second inclined section 2f₂ has an inclination of an angle Ω relative to the longitudinal axis yy′ of the cylindrical cannula 2 which is comprised between 12 and 16 degrees.

The second inclined section 2f₂ has beveled edges 2h relative to the longitudinal axis yy′ and directed in the direction of the outside of the cylindrical cannula 2 in order to constitute a cutting central edge 2i.

It is noted that the connection between the first and the second inclined section 2f₁ and 2f₂ is realized by cutting lateral edges 2j extending in the direction of the center of the cylindrical cannula 2 and more particularly of the cutting central edge 2i.

Each cutting lateral edge 2j. is inclined by an angle a comprised between 15 and 20 degrees relative to the longitudinal axis yy′ as a function of the outside diameter of the cylindrical cannula 2.

It will be understood that the slender profile 2a of the cylindrical cannula 2 allows the surgeon to pierce the tissues of the patient in order to bring the surgical needle holder 1 in the proximity of the operating site. The outside diameter of the cylindrical cannula 2 has small dimensions, that is, lower than or equal to 3 millimeters for determining the smallest scar possible.

The surgeon takes and holds the surgical needle holder 1 between the fingers of his hand by means of the grasping handle 2b in a locked position like a syringe in order to exert sufficient force on the latter to traverse the tissues of the patient.

As soon as the surgical needle 1 has been introduced by the surgeon at the level of the operating site he withdraws the retention means 5 in order to free the needle pusher 4 from the cylindrical cannula 2.

The surgeon takes and holds the surgical needle holder 1 between the fingers of his hand by the gripping handle 2b like a syringe and presses on the tappet wheel 4b of the needle pusher 4 in order to move the latter into the cylindrical cannula 2 in order to push and extract the surgical needle 3 at the level of the opening 2k of the slender profile 2a of this cannula (FIG. 2C).

When the tappet wheel 4b of the needle pusher 4 is supported against the edge of the grasping handle 2b the surgeon is certain that the surgical needle 3 integral with its suture thread 3a has been sufficiently extracted from the cylindrical cannula 2 in order to be recovered with the aid of a known suture pincer for making the suture points.

For withdrawing the surgical needle 3 from the operating site the surgeon introduces the latter by means of the suture pincer into the opening 2k delimited by the slender profile 2a of the cylindrical cannula 2.

Therefore, by an elastic deformation of the surgical needle 3 the surgeon can introduce it into the opening 2k and make it penetrate into the cylindrical cannula 2, allowing its retraction from the operating site during the extraction of this cannula.

Furthermore, it should be understood that the previous specification was given only by way of example and does not limit in any manner the scope of the invention which is not departed from by replacing the details of execution described by any other equivalent.

1. A surgical needle holder (1) allowing the guiding and the freeing of a surgical needle (3) integral with its suture thread (3a) into and from an operating site, which comprises a cylindrical cannula (2) holding the surgical needle (3) along a longitudinal axis yy′ and in a basically elongated position, which cannula (2) comprises a first end presenting a slender profile (2a) which ensures the traversing of the tissues and its penetration to the level of the operating site and comprises a second end opposite the first one integral with a gripping handle (2b), and in that this needle holder (1) comprises a needle pusher (4) introduced inside the cylindrical cannula (2) and retention means (5) that allows the needle pusher (4) to be temporarily held in a determined position relative to this cylindrical cannula (2).

2. The surgical needle holder according to claim 1, wherein the slender profile (2a) of the cylindrical cannula is constituted on a part of the circumference of this cannula by at least one inclined section (2f) whereas the other part of the circumference of the cannula is formed by a curved profile (2g) which approaches the longitudinal axis yy′.

3. The surgical needle holder according to claim 1, wherein the inclined section (2f) has an inclination at an angle β relative to the longitudinal axis yy′ of the cylindrical cannula (2) which is comprised between 10 and 16 degrees.

4. The surgical needle holder according to claim 1, wherein the curved profile (2g) has a radius of curvature R comprised between 7 and 10 millimeters.

5. The surgical needle holder according to claim 2, wherein the inclined section (2f) of the slender profile (2a) is realized over its length by at least two inclined sections (2f₁ and 2f₂) with different inclinations relative to the longitudinal axis yy′ of this cylindrical cannula (2).

6. The surgical needle holder according to claim 5, wherein the first inclined section (2f₁) extends from the peripheral edge of the cylindrical cannula (2) over a length of approximately 3 millimeters and in the direction of the free end of the cannula.

7. The surgical needle holder according to claim 5, wherein the first inclined section (2f₁) has an inclination of an angle δ relative to the longitudinal axis yy′ of the cylindrical cannula (2) which is comprised between 8 and 10 degrees.

8. The surgical needle holder according to claim 5, wherein the second inclined section (2f₂) is situated in the extension of the first inclined section (2f₁) and terminating at the level of the free end of the cylindrical cannula (2).

9. The surgical needle holder according to claim 5, wherein the second inclined section (2f₂) has an angle Ω relative to the longitudinal axis yy′ of the cylindrical cannula (2) which is comprised between 12 and 16 degrees.

10. The surgical needle holder according to claim 5, wherein the second inclined section (2f₂) comprises, relative to the longitudinal axis yy′, beveled edges (2h) directed in the direction of the outside of the cylindrical cannula (2) in order to constitute a central cutting edge (2i).

11. The surgical needle holder according to claim 10, wherein the connection between the first and the second inclined sections (2f₁ and 2f₂) is realized by cutting lateral edges (2j) extending in the direction of the center of the cylindrical cannula (2) and more particularly of the cutting central edge (2i).

12. The surgical needle holder according to claim 7, wherein each cutting lateral edge (2j) is inclined by an angle a comprised between 15 and 20 degrees relative to the longitudinal axis yy′.

13. The surgical needle holder according to claim 1, wherein the gripping handle (2b) comprises two branches (2c, 2d) extending in two opposite directions and perpendicular to the longitudinal axis yy′.

14. The surgical needle holder according to claim 1, wherein the needle pusher (4) is constituted by a cylindrical shaft (4a) comprising an internal bore (4g) which allows, during the placing of the surgical needle (3) in the cylindrical cannula (2), the housing of the suture thread (3a) and of a tappet wheel (4b) fixed to one of the ends of this cylindrical shaft.

15. The surgical needle holder according to claim 14, wherein the internal bore (4g) of the cylindrical shaft (4a) comprises opposite the tappet wheel (4b) a free end formed by an internal beveled edge (4d) with a conical profile which ensures on the one hand the introduction of the suture thread (3a) inside this shaft and on the other hand the guiding of the surgical needle (3) in the axis yy′ of the cylindrical cannula (2).
